(19) United States
(12) Patent Application Publication
(10) Pub. No.: US 2024/0226771 A9
(43) Pub. Date: Jul. 11, 2024

(54) Fluorinated Fluid Conditioning System

(71) Applicant: 3M INNOVATIVE PROPERTIES COMPANY, St. Paul, MN (US)

(72) Inventors: Tyler S. Matthews, Apple Valley, MN (US); Phillip E. Tuma, Faribault, MN (US); Kevin A. Frankel, Woodbury, MN (US)

(21) Appl. No.: 18/547,901

(22) PCT Filed: Mar. 9, 2022
(86) PCT No.: PCT/IB2022/052123

§ 371 Date: Aug. 25, 2023

Related U.S. Application Data
(60) Provisional application No. 63/160,536, filed on Mar. 12, 2021.

(51) Int. Cl. B01D 15/20; C07C 45/78; C07C 315/06

(52) U.S. Cl. CPC B01D 15/20; C07C 45/786; C07C 315/06

(57) ABSTRACT

Fluorinated fluid conditioning systems are described. In particular, fluorinated fluid conditioning systems including an electrically non-conductive fluorinated fluid and a filter including desensitized activated carbon sorbent are described.

### BACKGROUND

Fluorinated fluids are used in many different applications. In some systems where fluids are recycled within a system, the conditioning (including filtration) of such fluids is necessary to maintain the fluid in optimal condition by, for example, removing contaminants and degradation products.

### SUMMARY

In one aspect, the present description relates to a fluid conditioning system. The fluid conditioning system includes an electrically non-conductive fluorinated fluid and a filter including desensitized activated carbon sorbent. The electrically non-conductive fluorinated fluid is in fluid communication with the filter, where electrically non-conduct means greater than 25 kV breakdown strength at a 2.5 mm gap according to ASTM D877 (1987).

In another aspect, the present description relates to a method of preparing a fluid conditioning system. The method includes providing activated carbon sorbent, desensitizing the activated carbon sorbent, placing the desensitized activated carbon sorbent into a filter housing, and placing the filter housing including the desensitized activated carbon sorbent in fluid communication with an electrically non-conductive fluorinated fluid.

In yet another aspect, the present description relates to a method of filtering fluorinated fluid. The method includes providing desensitized activated carbon sorbent, providing an electrically non-conductive fluorinated fluid, and introducing the desensitized activated carbon sorbent to the electrically non-conductive fluorinated fluid, such that the desensitized activated carbon sorbent is in fluid communication with the electrically non-conductive fluorinated fluid.

### BRIEF DESCRIPTION OF THE DRAWINGS

FIG. 1 is a schematic side elevation cross section of an exemplary fluid conditioning system.

FIG. 2 is a flow chart showing an exemplary method of preparing a fluid conditioning system.

FIG. 3 is a flow chart showing an exemplary method of filtering fluorinated fluid.

### DETAILED DESCRIPTION

As used herein, “fluoro-” (for example, in reference to a group or moiety, such as in the case of “fluoroalkylene” or “fluoroalkyl” or “fluorocarbon”) or “fluorinated” means (i) partially fluorinated such that there is at least one carbon-bonded hydrogen atom, or (ii) perfluorinated.

As used herein, “perfluoro-” (for example, in reference to a group or moiety, such as in the case of “perfluoroalkylene” or “perfluoroalkyl” or “perfluorocarbon”) or “perfluorinated” means completely fluorinated such that, except as may be otherwise indicated, any carbon-bonded hydrogens are replaced by fluorine atoms.

As used herein, “fluid” refers to the liquid phase and/or the gaseous phase.

Fluorinated materials, including fluorinated fluids (fluid including both liquid and gas phases), are useful in numerous applications. This is at least in part to its electric non-conductivity at normal operating temperatures and environments, and its ability to prevent electrical short circuits (shorts). Electrical non-conductivity may be quantified by its breakdown strength at a given voltage, for a standard gap. For example, electrically non-conductive may mean greater than a 35 kV breakdown strength at a 2.5 mm gap, according to ASTM D877 (1987). In some embodiments, electrically non-conductive may mean greater than a 25 kV breakdown strength at a 2.5 mm gap, according to ASTM D877 (1987). In some embodiments, electrically non-conductive may mean having very high electrical resistivity, such as greater than 10∂Q cm.

The insulating properties of such fluorinated materials may make them particularly useful for insulating gasses (such as in electrical grid equipment) to prevent arcing electrical shorts, in fire-suppression applications to flood (and thereby remove heat or cut off oxygen from) electronic equipment without shorting it, and in immersion heat-transfer applications to efficiently transfer heat from electronic equipment without providing an electrically conductive medium to potentially allow short circuits to damage it.

For applications where fluorinated materials are in prolonged contact with electrical equipment, e.g., for insulating gasses or for immersion heat-transfer (cooling), it may be particularly important to avoid transfer of contaminants from the electrical systems into the fluid system. Accordingly, a conditioning system may be used to treat the fluid, filter the fluid, or otherwise remove or neutralize (or passivate) contaminants from the fluid.

Fluorinated materials having low global warming potential (GWP) are of particular interest for users of immersion cooling systems. Fluoroketones are an example of a class of materials that have low GWP and may be particularly suitable for immersion cooling. However, the same functional groups that enable the quick atmospheric breakdown (and thereby a low GWP) may also react with other elements within the immersion cooling system, including electronic components immersed therein, forming undesirable byproducts. For example, fluoroketones may undergo hydrolysis in the presence of water to form a highly corrosive acid (perfluoropropionic acid, or PFPA), which may circulate through the system and corrode or etch materials in contact with the circulating fluid. Along with PFPA, HFC-227ea (1,1,1,2,3,3,3 heptafluoropropane) is produced, which is a gas at room temperature. Because the gas may leave the system, such a process may be irreversible.

Certain fluorinated materials, such as fluoroketones, may also react with various organic molecules extracted from electronic components (for example, a —OH-containing plasticizer used in a polyvinyl chloride wire cladding) to form partially fluorinated esters and other degradation products. Partially fluorinated esters may also have considerably greater solubility in the overall fluorinated fluid system compared to the —OH molecules that preceded it, which makes filtration even more difficult.

Perfluorinated sulfone are another class of fluorinated fluids that, like fluoroketones, can react with water and other various organic molecules, albeit at a slower rate, to form corrosive or highly soluble degradation products.

Besides being corrosive, certain contaminants and degradation and reaction products in fluorinated fluid systems may act as a co-solvent for other potentially harmful contaminants, such as solder flux residue, plasticizers, metal salts, and (other) degradation and reaction products. Moreover, these contaminants may compromise other properties of the fluid, including dielectric properties.

Activated carbon is well-known as a sorbent used in filtration systems. However, its usefulness in other systems, e.g., for drinking water filtration, do not necessarily provide for a close analogue in fluorinated fluid systems. In fact, residual water on the surface of activated carbon can react with certain fluorinated fluids (e.g., fluoroketones). Likewise, activated carbon is chemically or catalytically active, which can enable or exacerbate certain of the degradation and the other reactions described elsewhere.

In some embodiments, the fluid described herein may be or include one or more fluoroketones. In some embodiments, the fluoroketones may be perfluorinated. In some embodiments, the fluoroketones may include from 5 to 12 carbon atoms or from 5 to 8 carbon atoms. In some embodiments, fluoroketones may be present in the fluid in an amount of a least 80 wt. %, at least 90 wt. %, or at least 95 wt. %, based on the total weight of the fluid.

In some embodiments, the fluid described herein may be or include perfluorinated sulfones. In some embodiments, the perfluorinated sulfones may include from 3 to 7 carbon atoms or from 4 to 6 carbon atoms. In some embodiments, perfluorinated sulfones may be present in the fluid in an amount of at least 80 wt. %, at least 90 wt. %, or at least 95 wt. %, based on the total weight of the fluid.

In some embodiments, in addition to or in place of the fluoroketones or perfluorinated sulfones, the fluids may also include (individually or in any combination): ethers, alkanes, perfluoroalkenes, alkenes, hydrofluoroalkenes, aromatic fluoroalkenyl esters, haloalkenes, perfluorocarbons, perfluorinated tertiary amines (saturated or mono-unsaturated), perfluoroethers (saturated or mono-unsaturated), cycloalkanes, esters, oxiranes, aromatics, siloxanes, hydrochlorocarbons, hydrochlorofluorocarbons, hydrofluorocarbons, hydrofluoroolefins, hydrochloroolefins, hydrochlorofluoroolefins, or hydrofluoroethers. Such additional components can be chosen to modify or enhance the properties of a fluid for a particular use.

In some embodiments, the fluids of the present disclosure may be hydrophobic, relatively chemically unreactive, and thermally stable. The fluids may have a low environmental impact. In this regard, the fluids of the present disclosure may have a zero, or near zero, ozone depletion potential (ODP) and a global warming potential (GWP, 100-year ITH) of less than 800, 500, 300, 200, 100 or less than 10. In some embodiments, the fluids of the present disclosure may have certain useful boiling points, for example, a boiling point between 30° C. and 75° C., or a boiling point between 130° C. and 150° C. In some embodiments, the fluids of the present disclosure may have a low dielectric constant, such as less than 4, or less than 2 at 1 kHz.

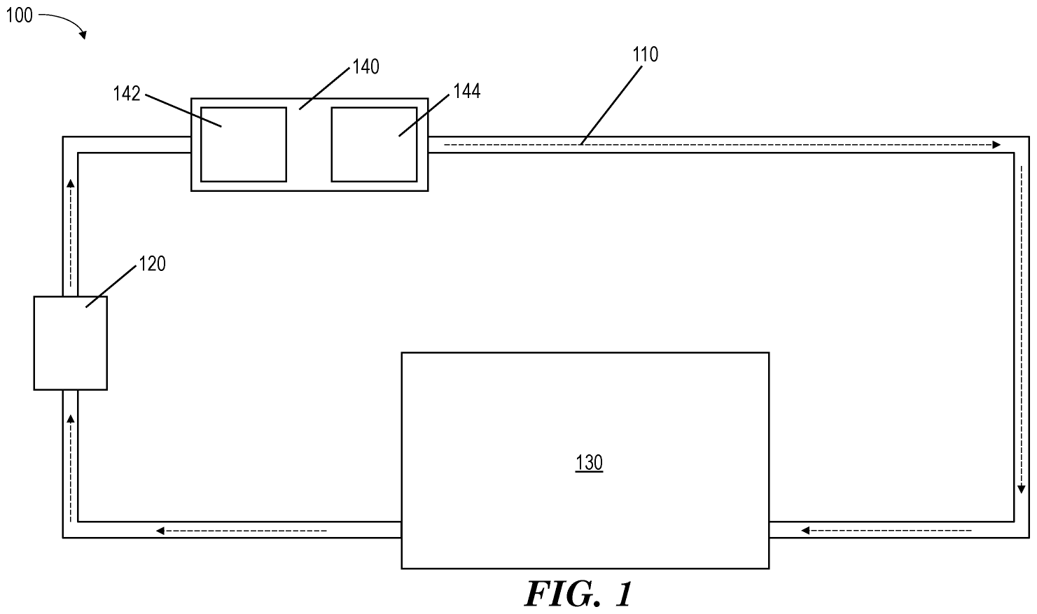

FIG. 1 is a schematic side elevation cross section of an exemplary fluid conditioning system. System 100 includes a fluid path 110, pump 120, container 130, and filter housing 140 including first sorbent 142 and optionally second sorbent 144.

Fluid path 110 may be any suitable conduit for appropriate fluids to travel within and through the fluid conditioning system. Fluid path 110 may vary depending on the matter phases of the fluid. For example, fluid path 110 may be formed or created by piping or tubing. In some embodiments, fluid path 110 may be formed by ducting. In some embodiments, and differently from the illustration in FIG. 1, fluid path 110 may be less rigidly constrained: for example, the fluid path 110 may be dictated by currents or other influences of force or pressure. In some embodiments, the fluid path 110 may emerge from an expansion and compression cycle, a phase change cycle, or from a heating and cooling cycle. As FIG. 1 is a schematic, real-world and practical embodiments may not include such a well-defined fluid path, may not have sharp, right angle turns, and may generally be in any suitable shape, form, and use any suitable material. The dashed arrow in FIG. 1 shown within the fluid path illustrates the general or dominant direction of flow (not illustrating reversals, cooling paths through a heat sink, or eddies or other regions of turbulent flow). The fluid path may constrain the fluid in some portions of the fluid conditioning system for portions of the fluid conditioning system, but not necessarily all of the system (for example, the fluid may be not be constrained by fluid path 130 while in container 130.

Pump 120 may be any suitable device or mechanism to circulate the fluid through the fluid conditioning system. Pump 120 may be a conventional fan or turbine to mechanically move fluid within fluid path 110. Pump 120 may be of any suitable style and its performance and characteristics may be selected based on the particular application and environment. Pump 120 may be powered by any suitable means, which are not illustrated in detail in FIG. 1. Pump 120 may not always be on, and may be intermittently triggered by a sensor or timer detecting a certain property threshold or the passing of an appropriate time interval.

Container 130 may be any suitable space or volume, which may be fully or partially filled with the fluid of fluid conditioning system 100. For example, container 130 may be a tank in which servers, computers, and/or other electronics are fully or partially immersed in a liquid fluorinated fluid, for heat transfer applications. In some embodiments, container 130 may be a switchgear or other sensitive electrical device wherein a gaseous fluid is present in a sufficient quantity to prevent the arcing between high voltage components. In some embodiments, container 130 may be filled with both liquid and gaseous fluid. In some embodiments, container 130 may not always include a fluorinated fluid, but it may be filled in response to a certain event (e.g., fire suppression in response to a detected fire or fire risk). Container 130 may contain any suitable additional components to manage the pressure or ambient environment within the container. Container 130 may also contain (not illustrated) components selected for the particular application (e.g., server docks, electrical and network cables, circuit breakers, etc.), or other systems not directly related to the fluid conditioning. For example, a heat exchanger, compressor, or other thermal management device, or a system to be able to safely access and repair or install/remove components within container 130 (with or without human interaction or intervention: for example, a fully or partially automated robotic arm system). Container 130, in FIG. 1, is illustrated as a discrete portion of fluid conditioning system 100, however, this is a depiction primarily for the ease of explanation, and certain components of the fluid conditioning system may be attached to or even present within container 130.

Filter housing 140 may be any suitable filter housing and may include suitable filtration or sorbent media. Depending on the application, filter housing 140 may incorporate or utilize different flow schemes within, in order to place the fluid to be conditioned in contact with the appropriate media. Suitable housings and general filtration regimes will depend on the particular requirements of the applications (e.g., liquid versus gaseous filtration, acceptable pressure drop, nature and quantity of the expected contaminants, etc.). Filter housing 140 may be specifically configured in order to allow for the quick and easy change of any filtration/sorbent media. Note, that for the purposes of this description, filtration is not limited to the separation of solid phases (not dissolved) and liquid phases. Filtration as used herein includes separation of the bulk fluid from dissolved impurities, degradation or reaction products, or other undesired contaminants, whether or not dissolved or of the same phase.

In FIG. 1, an exemplary first sorbent 142 and an optional second sorbent 144 are depicted. First sorbent may be any suitable material provided at the appropriate fill mass and density for the particular application.

In some embodiments, first sorbent 142 may be or include desensitized activated carbon. Desensitized activated carbon, also known as inert, dead, or passivated activated carbon, is activated carbon where surface oxides and other functional groups are removed or substantially removed, thereby making the sorbent material hydrophobic and catalytically inactive. In some examples, desensitized activated carbon may be helpful in significantly reducing the ability of the activated carbon sorbent (using standard activated carbon as a comparison) to chemically or catalytically react with fluorinated fluids to form undesirable products and, in particular, highly corrosive acids. In some embodiments, the desensitized activated carbon has also been dried in order to drive off any residual water content.

The particle/granule size and porosity of the desensitized activated carbon may be selected based on the particular application. In some embodiments, the desensitized activated carbon may be microporous, mesoporous, or macroporous, or may contain a combination thereof.

IUPAC has developed standard nomenclature for micro-, meso-, and macro-porosity. The term microporous refers to sorbents with pore diameters less than about 2 nm. The term mesoporous refers to sorbents with pore diameters greater than about 2 nm and less than about 50 nm. The term macroporous refers to sorbents with pore diameters greater than about 50 nm.

The desensitization or passivation process involves exposing the activated carbon to a de-oxidizing gas such as nitrogen or hydrogen at elevated temperatures (e.g. 700° C. to 1200° C.) to render the surface of the carbon free of oxygen-containing species, cooling the carbon under a de-oxidizing gas to a temperature of 30° C. to 500° C., contacting the carbon with a stabilizing or passivating substance such as ethylene, and further cooling to room temperature while in a de-oxidizing gas such as nitrogen. Such an exemplary process is described in U.S. Pat. No. 4,978,650 (Coughlin et al.).

When in contact with particular fluorinated fluids, for example, NOVEC 649 engineered fluid, available from 3M Company, St. Paul, Minn., (a fluoroketone) it has been observed that standard, commercially available activated carbon will produce a significantly higher level (e.g., 2 to 3 times or greater) of reaction products such as HFC-227ea as compared to the fluid without exposure to such a sorbent, where it is present but in trace amounts. Generation of such reaction products may be observed through nuclear magnetic resonance spectroscopy (NMR spectroscopy or simply NMR). Desensitized activated carbon produces significantly smaller increases in reaction products when exposed to NOVEC 649 and may in some cases be indistinguishable from the fluid without sorbent exposure.

In some embodiments, the suitable desensitized carbon mass necessary to achieve a desired contaminant level may be estimated based upon the measured adsorption isotherms of representative contaminants on the carbon and the anticipated organic burden in the container (e.g., an immersion cooling tank or other volume). This burden may be determined empirically or at least estimated by measuring or calculating extractable hydrocarbon levels in the polymers found or potentially found within the system, and tabulating the mass of contaminant in each. The desired filtration time constant (fluid mass/mass flow) dictates the mass flow through the filter. This time constant should be balanced with the residence time of the fluid within the filtration media (sorbent), which must be large enough to prevent breakthrough of contaminants which results in elevated contaminant levels and fouling of any additional filter media downstream of the carbon. The above method is merely exemplary and, of course, different optimizations, properties, and constraints should be considered depending on the specific application.

Optionally, filter housing 140 may include second sorbent 144. In some embodiments, second sorbent 144 may perform a different filtration/sorption function than first sorbent 142. In some embodiments, second sorbent 144 may be or include activated alumina. In some embodiments, the activated alumina may be pH-neutralized to remove hydroxyl groups. Suitable pH-neutralization methods may include, but are not limited to, water washing and subsequent drying or reactivation. Using activated alumina as a second sorbent may aid in removing acidic degradation products present in the fluid conditioning system. In some embodiments, the first and second sorbents may be present within the same filter housing. In some embodiments (not illustrated), the first and second sorbents may be present in separate filters or filter housings.

Other sorbents may be suitable for substitution or combination with either the first or (optionally) second sorbent, such as silica or other oxides of main group elements.

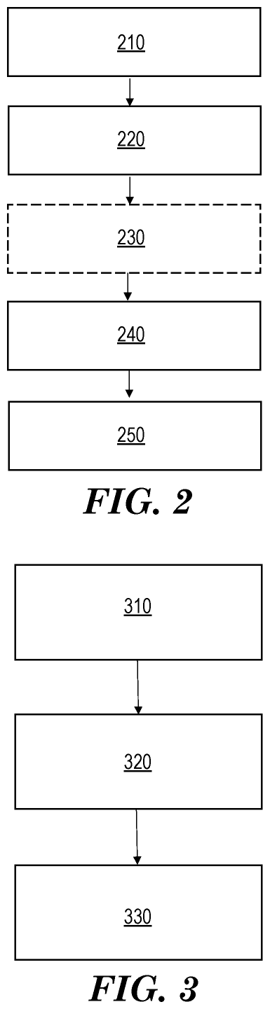

FIG. 2 is a flow chart showing an exemplary method of preparing a fluid conditioning system. The method includes the steps of 210, providing activated carbon sorbent material, optionally, 220, desensitizing the activated carbon sorbent material, 230, optionally removing residual water from the desensitized activated carbon sorbent material, 240, placing the desensitized activated carbon sorbent into a filter housing, and 250, placing the filter housing including the desensitized activated carbon sorbent in fluid communication with an electrically non-conductive fluorinated fluid.

FIG. 3 is a flow chart showing an exemplary method of filtering fluorinated fluid. The method includes the steps of 310, providing desensitized activated carbon sorbent material, 320, providing an electrically non-conductive fluorinated fluid, and 330, introducing the desensitized activated carbon sorbent to the electrically non-conductive fluorinated fluid, such that the desensitized activated carbon sorbent is in fluid communication with the electrically non-conductive fluorinated fluid.

### EXAMPLES

Materials Used in the Examples

| Abbreviation | Description and Source |
|---|---|
| NOVEC 649 | 1,1,1,2,2,4,5,5,5-Nonafluoro-4(trifluoromethyl)-3-pentanone, |
| | available under the trader designation “3M NOVEC 649 |
| | ENGINEERED FLUID”, from 3M Company, St. Paul, MN. |
| Kuraray GC 10 × 20 | Activated carbon with 10-20 μm standard particle size, available under |
| | the trade designation “KURARAY COAL ACTIVATED CARBON”” |
| | from Kuraray Co., Ltd., Tokyo, Japan. |
| Kuraray BGX | Cellulose-derived activated carbon available under the trade |
| | designation “KURARAY BGX”, from Kuraray Co., Ltd. |
| Kuraray RB | Charcoal-derived activated carbon available under the trade |
| | designation “KURARAY RB”, from Kuraray Co., Ltd. |
| Norit ROX 0.8 | Extruded activated carbon, available under the trade designation |
| | “CABOT NORIT ROX” from Cabot Corporation, Alpharetta, GA. |

### Adsorbent Drying Method

The adsorbent materials (i.e. the activated carbons), treated and untreated, were first dried in a N₂ inert furnace (available under the trade designation “BLUE M BOX FURNACE ATMOSPHERIC RETORT”, from Lindberg MPH, Riverside, MI). Approximately 50 grams of each adsorbent material was added to separate glass jars which were placed in a muffle furnace and the door was closed. Nitrogen flow was set to 90 standard cubic feet per hour (SCFH) and the furnace was purged for 10 mins. The nitrogen flow was then reduced to 20 SCFH, and the furnace was heated to 180° C. Drying was conducted at 180° C. for 16 hours. The jars were taken out of the oven at 180° C., capped and then allowed to return to room temperature. The samples were then placed in a dry N₂ box for storage.

### Adsorbent Material Treatment Method

The dried Kuraray BGX and Kuraray RB activated carbon adsorbents were further treated using a process described by Coughlin et al. in U.S. Pat. No. 4,978,650, which is incorporated herein by reference in its entirety. This process involves exposing the activated carbon to a de-oxidizing gas such as nitrogen or hydrogen at elevated temperatures (e.g. 700° C. to 1200° C.) to render the surface of the carbon free of oxygen-containing species, cooling the carbon under a de-oxidizing gas to a temperature of 30° C. to 500° C., contacting the carbon with a stabilizing or passivating substance such as ethylene, and further cooling to room temperature while in a de-oxidizing gas such as nitrogen.

Compatibility of Adsorbents with NOVEC 649

One gram of the chosen adsorbent material (after all drying and treatment methods) was weighed out and added into a 30 ml poly bottle container, 10 ml of NOVEC 649 fluid was then dispensed into the container, a cap applied, and 3M vinyl tape wrapped around the cap to prevent accidental leakage. Finally, the container was placed on a rolling mill and mixed for 24 hours at room temperature. The bottles were set aside for at least 24 hours to allow for the adsorbent to settle out. A syringe was used to remove 3-4 ml of the supernatant fluid from the bottle and dispensed into an 8-ml poly bottle.

The liquid sample in the 8-ml poly bottle was analyzed with NMR to look for indication of reaction with the NOVEC 649. The ¹H-NMR and ¹⁹F-NMR test methods were as follows. Neat aliquots (˜0.8-1.0 mL) of the NOVEC 649 sample fluids that were treated with the adsorbent material were transferred into pre-dried 5-mm outer diameter glass NMR tubes in a nitrogen-purged glove box. No extra standards of any kind were added to the sample fluids to avoid introduction of trace amounts of extra water. Initial 600.1 MHz ¹H-NMR spectra and 564.7 MHz ¹⁹F-NMR spectra were acquired using a Bruker Avance-III HD 600 FT-NMR spectrometer (MAID #1467) that was operating with a helium cooled 5-mm inverse-detection gradient TCI cryoprobe (MAID 1472) at an analysis temperature of 25° C. The HFC-227ea impurity was used as the ¹H/¹⁹F cross integration standard in place of the usual 1,4-bis(trifluoromethyl)benzene (p-HFX) standard to permit the cross correlation of the relative ¹H and ¹⁹F signal intensities for quantitative purposes.

When NOVEC 649 reacted with an adsorbent, one by-product is 1,1,1,2,3,3,3 heptafluoropropane (CF₃—CFH—CF₃), designated as HFC-227ea, a colorless gas. Another by-product was hexafluoropropylene (CF₃—CF═CF₂), designated as HFP, another colorless gas. NOVEC 649 in the neat form contained a low level of HFC 227ea gas, designated by the symbol “(O)” in Table 1 (below), and typically has concentrations of HFP below the detection limit, designated by the symbol “(O)” Table 1. When these gases are present at levels ≥2-3 times the concentration of the neat fluid, this was an indication of reactivity of the adsorbent with the NOVEC 649, which was designated by the symbol “(X)” in Table 1.

The ¹⁹F-NMR and ¹H-NMR spectra were used to measure the absolute weight percent concentrations of HFC-227ea and hexafluoropropylene (CF₃—CF═CF₂), HFP, in the NOVEC 649 samples treated with adsorbent material. The qualitative and quantitative compositional results that were derived from the single trial ¹⁹F/¹H-NMR cross integration spectral analyses are summarized in Table 1.

TABLE 1
Compatibility of Adsorbent Materials with NOVEC 649

| | | HFC 227ea | HFP |
|---|---|---|---|
| | Material | By-Product | By-Product |
| | Neat-NOVEC 649 | (O) | (O) |
| Comparative | Kuraray GC 10 × 20 | (X) | (O) |
| Example 1 | | | |
| Example 2 | Kuraray BGX (treated) | (O) | (O) |
| Example 3 | Kuraray RB (treated) | (O) | (O) |
| Comparative | Norit ROX 0.8 | (X) | (O) |
| Example 4 | | | |

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modifications and variations of the concepts herein disclosed may be resorted to by those of ordinary skill in the art, and that such modifications and variations are considered to be within the scope of embodiments of the present invention.

What is claimed is:

1. A fluid conditioning system, comprising:
an electrically non-conductive fluorinated fluid;
a filter including desensitized activated carbon sorbent;
wherein the electrically non-conductive fluorinated fluid is in fluid communication with the filter;
wherein electrically non-conductive means greater than 25 kV breakdown strength at a 2.5 mm gap according to ASTM D877 (1987).

2. The fluid conditioning system of claim 1, wherein the electrically non-conductive fluorinated fluid has a GWP of less than 100.

3. The fluid conditioning system of claim 1, wherein the electrically non-conductive fluorinated fluid has a boiling point between 30° C. and 75° C.

4. The fluid conditioning system of claim 1, wherein the electrically non-conductive fluorinated fluid has a boiling point between 120° C. and 150° C.

5. The fluid conditioning system of claim 1, wherein the electrically non-conductive fluorinated fluid has a dielectric constant at 1 kHz of less than 4.

6. The fluid conditioning system of claim 1, wherein the electrically non-conductive fluorinated fluid is a fluoroketone.

7. The fluid conditioning system of claim 1, wherein the electrically non-conductive fluorinated fluid is a perfluorinated sulfone.

8. The fluid conditioning system of claim 1, further comprising a pump for circulating the electrically non-conductive fluorinated fluid through the filter.

9. The fluid conditioning system of claim 8, further comprising a secondary filter in fluid communication with the electrically non-conductive fluorinated fluid.

10. The fluid conditioning system of claim 9, further comprising a filter housing, wherein the filter and the secondary filter are both within the filter housing.

11. The fluid conditioning system of claim 9, wherein the secondary filter includes activated alumina.

12. The fluid conditioning system of claim 11, wherein the activated alumina has been pH neutralized.

13. The fluid conditioning system of claim 11, wherein the secondary filter is downstream from the filter.

14. A method of preparing a fluid conditioning system, comprising:
providing activated carbon sorbent;
desensitizing the activated carbon sorbent;
placing the desensitized activated carbon sorbent into a filter housing;
placing the filter housing including the desensitized activated carbon sorbent in fluid communication with an electrically non-conductive fluorinated fluid.

15. The method of claim 14, further comprising the step of removing residual water from the desensitized activated carbon sorbent.

16. The method of claim 14, wherein the electrically non-conductive fluorinated fluid is a fluoroketone.

17. The method of claim 14, wherein the electrically non-conductive fluorinated fluid is a perfluorinated sulfone.

18. A method of filtering fluorinated fluid, comprising:
providing desensitized activated carbon sorbent;
providing an electrically non-conductive fluorinated fluid;
introducing the desensitized activated carbon sorbent to the electrically non-conductive fluorinated fluid, such that the desensitized activated carbon sorbent is in fluid communication with the electrically non-conductive fluorinated fluid.

19. The method of claim 18, wherein the electrically non-conductive fluorinated fluid is a fluoroketone.

20. The method of claim 18, wherein the electrically non-conductive fluorinated fluid is a perfluorinated sulfone.
